SEQUENCE LISTING (7 sequences)

SEQ ID NO: 1 - DNA, 622 bp, Homo sapiens
actacattgagcatgatgtgtctcctgaatgtgtgtttcatgtgaagtattgttctgattaactgacatccttgcttacaagtttcactaaccctttggagtttaagcacaaatgcacaaagggaaaagaggacgacctgtttggggttcttttttgcaaaaacaaacagtcgcatgctggacgctaacaccaagcttacactgtgtgtgtgatacggctgagctgctccataaggctctatcttttatctgcccaaggcgtgccctgcaactctggaatgcagagcagttgctggggtgattgacctaggcacagtggagatatttcccatcttcaaagccatgcaaaagggcctccttgaccaagacacaggcctagtgcttctggaatctcaggttatcatgtctggcctcattgcccctgagacgggtgaaaacctctctttggaggagggcgtagccagaaacctcattaatccccagatgtaccagcagctccgggagctacaggagccctggccttaataagcaggcttactgagagcagaggccctctttctgtggtggaagcaattgaaaagacaataatcagtgagacagttggactgaaaatcttagaagt

SEQ ID NO: 2 - DNA, 268 bp, Homo sapiens
gtggtggtgggcgcctgtaatcccagctacttgggaggctgaggcagagaactgcttgaacccaggaggcagaggttacagtgagccaagatcgcaccactgcactccagcctccagccttcagccttggtgacagagcaagactctgtctcaaaaagaaagaaaaagaaaaagactgtgaaagaacacacatcaaaatgttaagcagtggtttgtatcttgaaggacaatttttttttattggaatgtttcttctctatatttttgg

SEQ ID NO: 3 - DNA, 496 bp, Homo sapiens
cgggcccgggatggactgaaccaagaccagcagccaacttagaggctcagttttaaggccttgacttgggatagtaagattagagatttccagcagtgtctcctccccgcacctccccccacccccccgccccccgctttttagtgaagagaaagtcacataaagataaccatttaaaagtgagtaattcaaggccaggcgcggtggcccatgcctgtaatcccagcactttgggcggctgaggcaggtggatcacttgaggttaggagttcgagcccagcctggtcaacatggtgaaaccccgtctctactaaaaatataaaaattagccgggtgtggtggcaggcacctgtaatcccagctattagggaggctgaggcaggagaattgcttgagcctgggaggcagaggttgcagcgagccaagattgtgccactgtactccagcctgagcgacggagcgagaatctgtctcaaaaaaaaaaaaagataattca

SEQ ID NO: 4 - DNA, 772 bp, Homo sapiens
agtttctaaggatcatgtctgcgagccaggattcccgatccagagacaatggccccgatgggatggagcccgaaggcgtcatcgaggtgagactggagaaatggaattctgtcctcccccattacaactttcagccgtatagagttagagtggcctcttgattgatttcccagatcatctagaagcagctggtttccctaaagggaggagggttgtaagctctgaggcttttgttagtaggcaccagattctgtttgctcggagactacagctcagctccaccttttccatgactcaagctttaatttctttgcatcccctagagtaactggaatgagattgttgacagctttgatgacatgaacctctcggagtcccttctccgtggcatctacgcctatggttttgagaagccctctgccatccagcagcgagccattctaccttgtatcaagggtgagacctctcagtcccagaagacattgtggactgtccctgacctgggtagagtggcatctggttggtgatgcccatctcatatcagccagggacaaagcaactccttgttcatcccagcttggcttttgatccgtgcccatgcctggttcatgccttggacacataggtttcctttaaagaggtggtattgtagccagcttatatttgcatctatagccatgtttctagtccagcttggtgtgcaatactagatgagttaataactggtccttgtttctgatctggttcccattgtgtaactgtgttgattggg

SEQ ID NO: 5 - DNA, 705 bp, Homo sapiens
gccttatggccggggacaaccttagccaaccatttacccaaataaagtataggcgatagaaattgaaacctggcgcaatagatatagtaccgcaagggaaagatgaaaaattatagccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacatcaaccaaca

SEQ ID NO: 6 - DNA, 301 bp, Homo sapiens
tggctcatggctacaatcccagcactttgggaggccgaggcaggcagatcaccggaggtcaggagttcaagaccagcctgaccaacacggagaaaccccgtcccaactaaaaatacaaaattagccaggcatggtggcacatgcctgtaataccagctactcaggaggctgaggcaggagaatgacttgaacctgagaggcaaatgctgcagtgagccgagatcaggccattgcactccagcctgggaaacaagaggcaaaactccgtctccaaaaaaaaaaaaaaaaaaaaaaaaaaaaa

SEQ ID NO: 7 - DNA, 953 bp, Homo sapiens
  misc_feature (354)..(354): n is a, c, g, or t
gatatgaaatgactccctcagacaatttttaaaaagataagttttttaaagaccaataaaaacctaagggacaaaataagacattgatgatttgaaatttctttgtaacaaaatatactataaatttgaaagcaaaggatagactggaagagaatatttgcaatatttaaaacaggctaatggtcagtgctcacaatatataacatgctctcatgtatcaattttaaaacaacacccttgtaaaaaaaaaaaaaaaaaggatccaatgaggcagggtacaaataacaaattcttagcaaaataatttagctcctgaaatgatactcattcttactggaaaccaggggaatgcanattctaataggttattttttttgcttatgaaatttgcaanaataaaagngactactgagcttcnttttttgtaanagngtagngaaactagtatctgcatncccngtnggggatggtataaattggcacagtattttttacattagngcattgatgtatttttaaaacacttatatattgcacaattatcaaatctgcacagcagtttttatttgataatctgttctacaaaaatactcataaaggacacaaatataaggaattacatcattaattattatcaattcccatgnagccatttaaaagcatttngggggatctctatggaaattggcatggaattatttnatttcanaaaaattatttttttaatccatggaanccttggatactggnttgcggggcttgaaaaacttcttccaagaaaattntttatttgggaaaaaaaattaaaggnaaaaatttgggaattaaataaggganttccatnataagggangggtaaaaacctaaaaaagccngggtngggggnatttttaatngggggttaanngggggattacnatttggnaaaaantttggnaanggggntttttntt

(19) United States
(12) Patent Application Publication
(10) Pub. No.: US 2005/0084869 A1
(43) Pub. Date: Apr. 21, 2005

(54) Novel composition and methods for the diagnosis of lung cancer

(76) Inventor: Sung Hyun Kim, Seoul (KR)

Correspondence Address: Sung Hyun Kim Ph.D., 25705 Chestnut Way, Stevenson Ranch, CA, 91381, US

(21) Appl. No.: 10/689,862

(22) Filed: Oct. 20, 2003

(51) Int. Cl. C12Q 1/68

(52) U.S. Cl. 435/6

(57) ABSTRACT

The present invention provides a method of identifying specific over-expressed lung cancer genes using a novel in-situ screening approach and further relates to a composition comprising novel genes that are identified using the present method. The present invention further contemplates providing a high-throughput molecular classification and diagnostics methodology based on detecting mRNAs for the identified lung-cancer-overexpressed genes.

### FIELD OF THE INVENTION

The present invention relates to a method of identifying genes that are overexpressed in different types of cancer cells, in particular, in lung cancer cells and further relates to a composition comprising novel genes that are identified using the present method. The present invention further concerns compiling the spatio-temporal expression profiles of the lung-cancer-overexpressed genes and establishing an algorithm that connects the gene expression profiles to the clinical phenotypes of various lung cancers. The present invention can be used as the primary tools for developing a high-throughput molecular classification and diagnostics methodology based on detecting mRNAs for the identified lung-cancer-overexpressed genes, or the protein products for them.

### BACKGROUND

Modern medical science is constantly searching for new and more powerful agents to prevent or treat cancer. Yet, despite the costs and efforts invested, cancer remains as a common cause of death throughout the world. Although revolutionary advances are being made in molecular and genomic medicine, no universally successful diagnosis and/or treatment is currently available for cancer. In particular, lung cancer is one of the primary causes of death among both men and women in the world. In 2000, approximately 160,000 deaths in USA, and 2.24 million deaths around the world were accounted for by lung cancer (1). There may not be an immediate solution for this deadly disease, but the first step has to be effective diagnosis, particularly during the early symptomatic stages when a variety of effective treatments are in fact available (2-8).

One of the most common diagnostic procedures for lung cancers are photographic methods such as X-ray or CT scan. These methods solely rely on tumor morphology or topography of lung, and therefore, cannot distinguish benign abnormalities that are frequently caused by various lung diseases, from malignancy. Furthermore, invasion or metastasis can occur as early as when tumor size is 1-2 mm in diameter, but it is impossible to detect such a small primary tumors, not to mention even smaller secondary tumors. Without knowing distant metastasis, surgery is often carried out, and as a consequence, recurrence rate is high, 15% in the first postoperative year alone (2). The recurrence rate is a sum of metastatic secondary tumors and independent cancer development. The recurrence from metastasis is expected to start shortly after the surgery, and to decrease as time passes, and the recurrence from independent carcinogenesis should remain constant. Indeed, postoperative recurrence rate among a large number of patients decreases progressively, but stops decreasing at 2% (8, 9, 10). This data indicate that the 2% of the recurrence rate is due to independent cancer development. Therefore, other than the 2% of new cancer development, majority of the recurrence cases seems to be caused by miss-diagnosis of metastasis. If the tumors had been diagnosed of metastatic nature even in the absence of apparent secondary tumors, an alternative therapy such as chemo or radiation therapy could be considered. Therefore, it is not only the early diagnosis, but also the nature of tumor that needs to be determined.

Sputum cytology has been the most effective method for early diagnosis since the first case report in 1951 (11-23). Sputum contains various epidermal cells from major bronchi, exfoliated when coughing. Each sample requires careful inspection under microscope by trained pathologist for the presence of cancer cells. For this method to be used for large scale population screening, it should be renovated into high-throughput automation. One of the most attractive aspects of sputum cytology is that it presents live cells as test material which can be used for molecular genetic diagnosis. Molecular diagnosis can also improve the test procedure into automated high-throughput methodology. Indeed, such attempts to use sputum samples for molecular genetic diagnosis are being made worldwide (24, 25).

However, of all the currently available markers, none has achieved sufficient diagnostic significance to reach clinical application. Sensitivity needs to be high because test samples contain very small number of cancer cells, and high specificity, because they are mixed with a large number of normal cells. For a genetic maker to be sensitive, it should be expressed at a high level, and to be specific, its expression in large number of normal cells must be very low. To discover lung cancer markers of this category, spatial expression pattern must be examined in situ to differentiate gene expression in cancer cells from normal cells within a tumor. Current screening methods including various differential screening strategies compare and subtract between normal versus tissue of interest such as cancer. These experiments have two intrinsic problems. First, they do not discriminate normal versus cancer cells within the tumor. Second, each experiment uses one specific type of cancer, and therefore is unable to detect lung cancer markers of different types and stages. As lung cancer is a collective term for epigenetically diverse group of cancers, there exist numerous molecular pathways and downstream target biomarkers. (26, 27, 28) Therefore, it is unlikely to discover single universal lung cancer marker capable of diagnosing all different types of lung cancer, but a group of markers and their combinatorial expression profiles can render the necessary specificity and sensitivity for a universal diagnostics.

An invention directed to a simple and accurate diagnosis of lung cancer in an early stage would be a significant improvement to existing therapies.

### SUMMARY OF THE INVENTION

The present invention provides a method for detecting lung cancer in early stages. More specifically, in one embodiment, the invention is directed to identifying genes that are overexpressed in lung cancer cells. One aspect of this embodiment is the method of identifying these genes. Another aspect of this embodiment is providing the identity of these novel genes and a composition comprising such novel genes. Still another aspect of this embodiment is to provide these lung cancer specific gene expression profiles which can be used as the primary tools for molecular classification and diagnostics.

Another aspect of this embodiment of the present invention is to develop algorithm that connects the gene expression profiles to the clinical phenotypes, such as cancer histotypes, developmental stages, responsiveness to various therapies, or even survival/death rates. This kind of molecular classification of various clinical phenotypes may help in designing a meaningful and effective therapy for individual patients.

In another embodiment of the present invention, the genes expression profiles and molecular classification can be used to develop a high throughput diagnostic methodology including mechanical hardware that can process multiple samples and generate reliable gene expression profiles to assign each sample to proper classes.

### BRIEF DESCRIPTION OF THE FIGURES

FIG. 1 is an illustration depicting certain limitations of conventional methods of photographic diagnosis of cancer.

FIG. 2 an exemplary illustration showing certain limitations of conventional sputum cytology and also the use of biomarker test as embodied in the present invention.

FIG. 3 is a chart illustrating both the conventional differential screening and the novel in-situ screening method to identify cancer specific gene markers according to one embodiment of the present invention.

FIG. 4 is a graphic illustration depicting the method of screening and identifying over-expressed lung cancer markers according to one embodiment of the present invention.

FIG. 5 is a diagram depicting a summary of exemplary experimental procedures according to one embodiment of the present invention.

FIG. 6 is a table summarizing new lung cancer markers identified according to the method in one embodiment of the present invention.

FIG. 7 illustrate the gene expression patterns of novel marker #4, eukaryotic translation initiation factor 4A, isoform 1 (EIF4A1) identified according to one embodiment of the present invention.

FIG. 8 is an exemplary illustration depicting how a specific gene or gene set can correlate to particular morphological and clinical phenotypes.

### DETAILED DESCRIPTION OF THE INVENTION

Certain terms, used in the context of describing the invention, are defined, and have the following meanings when used herein and in the appended claims.

### Lung Cancer

The term “lung cancer” generally refers to malignancies or tumor cells grown out of control in lungs. As a way of providing some background, researchers suggest that cancer cells show six essential alterations in cell physiology that collectively dictate malignant growth: self-sufficiency in growth signals; insensitivity to growth-inhibitory (antigrowth) signals; evasion of programmed cell death (apoptosis); limitless replicative potential; sustained angiogenesis; and tissue invasion and metastasis. Each of these physiologic changes—novel capabilities acquired during tumor development—represents how cancers avoid an anticancer defense mechanism hardwired into cells and tissues. These six features are shared in common by most, if not all, types of malignant tumors.

Also, lung cancers that begin in the lungs are divided into two major types, non-small cell lung cancer and small cell lung cancer, depending on how the cells look under a microscope. Each type of lung cancer grows and spreads in different ways and is treated differently. Small cell lung cancer accounts for about 20% of all lung cancers. Although the cancer cells are small, they can multiply quickly and form large tumors that can spread to the lymph nodes and to other organs such as the brain, the liver, and the bones. Smoking almost always causes this kind of cancer; it is very rare for someone who has never smoked to have small cell lung cancer.

Non-small cell lung cancer is the most common type of lung cancer, accounting for almost 80% of lung cancers. There are three major subtypes within this group. Squamous cell carcinoma is the most frequent types (60% of all lung cancers) that are highly linked to history of smoking. It tends to be found centrally, near major bronchi. Adenocarcinoma is constantly increasing and usually found in the outer region of the lung. Large-cell undifferentiated carcinoma can appear in any part of the lung and tends to grow and spread quickly, resulting in a poor outlook for the patient.

Other type of tumors can occur in the lungs. Some of these are not cancer (benign) and others are cancerous (malignant). Carcinoid tumors, for example are slow-growing and usually cured by surgery.

The general (nonspecific) signs and symptoms of lung cancer include: a cough that doesn't go away and gets worse over time, constant chest pain, couching up blood, shortness of breath, wheezing, or hoarseness, repeated problems with pneumonia or bronchitis, swelling of the neck and face, loss of appetite or weigh loss or fatigue, but often there is no obvious symptom so the patients are not aware of their cancer. Often, by the time some of those illustrated symptoms are detectable or noticed, cancer cells have already spread out to other parts of the body making it more difficult to treat.

Cancer treatment depends on a number of factors, including the type of lung cancer, the size, location, and extent of the tumor, and the general health of the patient. Many different treatments and combinations of treatments may be used to control lung cancer. Surgery is an operation to remove the cancer, and chemotherapy is the use of anticancer drugs to kill cancer cells throughout the body. Further, radiation therapy involved the use of high-energy rays to kill cancer cells, so it affects cancer cells in limited area. Photodynamic therapy (PDT), a type of laser therapy, involves the use of a special chemical that can remains in cancer cells. Nowadays, gene therapy using critical genes such as p16, p27 and IGF-I is expected to carry out as a part of new trials for cancer treatment.

Several causes of lung cancer have been discovered. A widely well known cause is the use of tobacco. Carcinogen, harmful substance, in tobacco damages the cell in the lungs. Over time, the damaged cells may become cancerous. The likelihood that a smoker will develop lung cancer is affected by the age at which smoking began, how long the person has smoked, the number of cigarettes smoked per day, and how deeply the smoker inhales.

There are some more substances known to cause lung cancer. These can cause damage to the lungs that may lead to lung cancer. Radon is an invisible, odorless, and tasteless radioactive gas that occurs naturally in soil and rock. Asbestos is the name of a group of minerals that occur naturally as fibers and are used in certain industries. Its fibers tend to break easily into particles. When the particles are inhaled, they can lodge in lung and damage cells. Further, exposure to certain air pollutants, such as by-products of the combustion of diesel and other fossil fuels may be related to lung cancer. It is also known that certain lung diseases, such as tuberculosis, increase a person's chance of developing lung cancer.

In addition, a person who has had lung cancer once is more likely to develop a second lung cancer compared with a person who has never had lung cancer. Further, certain oncogens or tumor suppressor genes have genetic susceptibility can cause lung cancer. Among these notorious genes, Ki-ras, Her2-neu and bcl-2 can cause NSCLC. Myc and c-kit are cause for SCLC. Still, critical genes not determined might involve the cause of lung cancer.

### Polynucleotide

As used herein, the term “polynucleotide” generally refers to a RNA or DNA molecule that has been isolated free of total genomic DNA of a particular species. Included within the term “polynucleotide” are RNA or DNA segments and smaller fragments of such segments, and also recombinant vectors, including, for example, plasmids, cosmids, phagemids, phage, viruses, and the like.

As will be understood by those skilled in the art, the polynucleotide segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

### Gene Expression

As used herein, the term “gene expression” refers to cellular process in which DNA is transcribed into RNA, processed to remove non-coding sequences, and transported to cytoplasm in the form called mRNA. The absolute amount of cytoplasmic mRNA serves as quantitative measurement of gene expression. Gene expression can be largely divided into activated and basal level. Basal level of transcription maintains approximately five copies of mRNA per cell, and activated transcription often generates thousands of mRNA molecules of a gene per cell. In normal lung, less than 10% of thirty thousand human genes are overexpressed (activated level), and the rest remain silent (basal level). Different batteries of genes are expressed to determine the kind and state of a tissue. In other words, brain is different from lung or liver in that it expresses brain genes instead of lung or liver genes, and vice versa. Nonetheless, as different tissues carry out same basic cellular metabolism, they share many common genes involved in the processes. It is estimated that among the 10%, only about 0.2% are tissue specific, and the rest are common. The small fraction of genes showing cancer cell specificity attracts a lot of scientific attention, as they may provide information or clues to the mechanism underlying tumor or genesis and molecular tools to diagnose and classify various types of cancer.

### Molecular Classification

The term “molecular Classification” refers to pathological procedure that categorizes clinical symptoms according to the genetic nature marked by expression of representative biomarkers. Conventional diagnosis relies heavily on histological features such as cellular morphology, as implied in names like small cell lung cancer, non-small cell lung cancer, squmous cell lung cancer, adenocarcinoma, and large cell carcinoma. The conventional classification seems not very efficient for the intended purpose. For instance, newly anticancer drug Iressa showed significant reduction of tumor size only in 13% of the patients tested. According to current classification, no close correlation was observed except that female smokers shows a bit better response to the drug. Theoretically, every symptom is generated as an end result of cooperative work of a battery of genes, and therefore, expression of these genes can serve as excellent markers for the corresponding symptoms.

Multiple carcinogenic pathways can transform a normal cell into cancer development. Those tumors arose from same type of cells but by different pathway might not show any morphological differences, but may exhibit different phenotypes such as responsiveness to an anti-cancer drug that interferes one pathway but not the others. Indeed, 2003 May FDA approved lung cancer drug Iressa based on clinical trial that showed high efficacy in 13% of the patients participated. Iressa is recommended for late stage patients as severe side effects including death was observed in some of the participants. This clinical data strongly suggest the existence of the predicted unidentified subgroups among conventional classification. Molecular classification is anticipated to further subdivide current categories, providing rich genetic parameters to which clinical symptoms can be correlated. If molecular classification helps sorting out the 13% who would respond positively before the chemotherapy, the remaining 87% need not take the life threatening chemotherapy. By the same token, molecular classification of metastatic tumors will recommand against surgical resection even in the absence of any visible secondary tumors. The molecular classification will not only renovate current diagnosis, but also generate precious information about carcinogenic.

### In Situ Hybridization

The term “in situ hybridization” refers to an experimental procedure that visualizes gene expression pattern within in situ spatial cellular context. In other words, it generates information regarding which genes are activated where and how, providing a solution to two major hurdles in lung cancer study, the intra- and inter-heterogeneity of lung tumors. Intra-heterogeneity refers to diversity of tissue within a tumor. Lung is an organ with various tissues organized in a highly regulated fashion. Malignant tumors are different from normal lung in that the tissue organization is lost due to unchecked growth of cancer cells, but the diversity of tissues still remains. Because of the non-cancerous tissues within a tumor, all biomarkers need to be confirmed for their cancer specificity by in situ hybridization. Inter-heterogeniety of tumors refers to diversity of tumors in their intrinsic nature. First of all, benign tumors are frequently found in lung particularly among those with the history of various lung disease such as tuberculosis. Secondly, malignancies are classified into two dozen types and as many stages. In addition, malignant tumors shows diversity in other parameters as growth rate, metastasis, responsiveness to chemotherapy, recurrence rate, and even death/survival rate. Despite its efficacy in determining tissue specificity, in situ hybridization has been limited in its usage because of low processivity, which is recently improved dramatically by tissue array technology. A tissue array is mounted with 60-240 different cancer samples on a histological slide to be processed just as one sample, allowing both spatial gene expression analysis within each tumor and comparative gene expression analysis among various tumors.

The present invention provides a method for detecting lung cancer in early stages. More specifically, in one embodiment, the invention is directed to identifying and expressing genes that are specific to lung cancer cells. One aspect of this embodiment is to provide these lung cancer specific gene profiles which can be used as the primary tools for molecular classification and diagnostics. Once these lung cancer specific genes are isolated, their gene expression patterns can be profiled by in situ hybridization utilizing techniques such as tissue array technology.

Another aspect of this embodiment of the present invention is to develop algorithm that connects the gene expression profiles to the clinical phenotypes, such as cancer histotypes, developmental stages, responsiveness to various therapies, or even survival/death rates. This kind of molecular classification may help in designing more educated, meaningful and effective therapy for individual patients.

In another embodiment of the present invention, the genes expression profiled and molecular classification can be used to develop a high throughput diagnostic methodology including mechanical hardware that can process multiple samples and generate reliable gene expression profiles to assign each sample to proper classes.

In one embodiment, the present invention applies molecular genetics to sputum cytology, such as DNA test of sputum samples, to provide a simple, safe and accurate detection of lung cancers in early stages.

Identification and Isolation of Over-Expressed Lung Cancer Specific Genes:

Purpose of the Invention

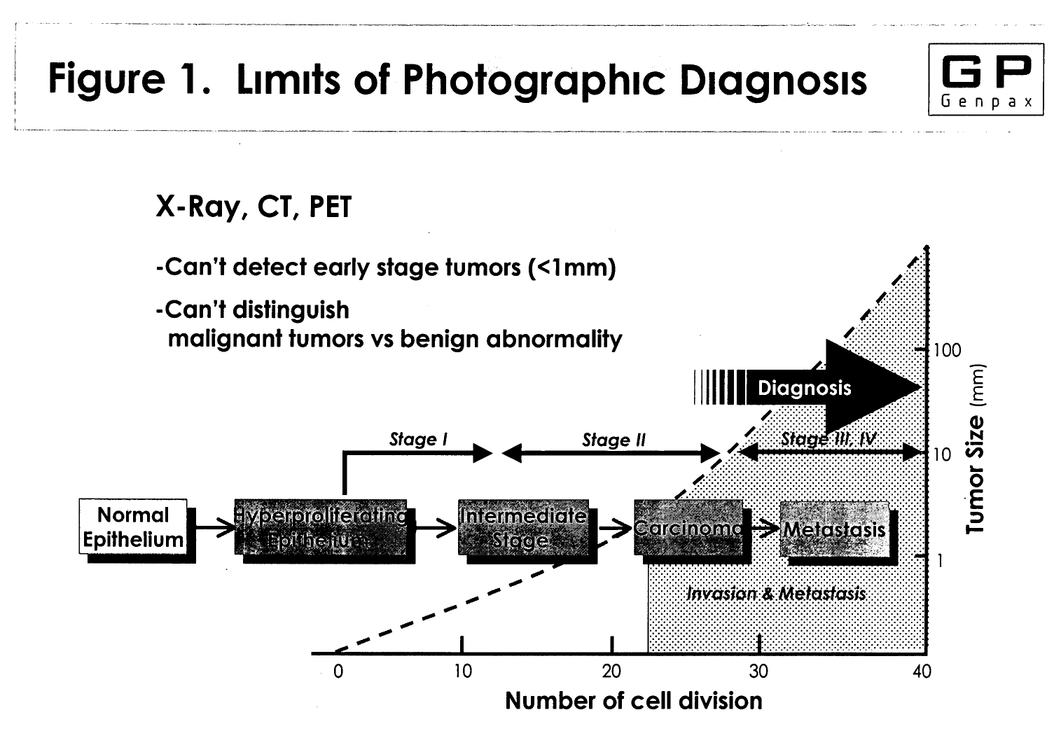

As illustrated in FIG. 1, conventional photographic diagnostic methods such as X-ray, computed tomography (CT), or PET rely on the morphology of tumors. However, a significant problem of such photographic methods is its size limitation. Chest X-ray can not detect tumors smaller than 10 mm, and even the most advanced computed tomography can not detect those smaller than 1 mm which is already big enough for invasion or metastasis to occur depending on tumors. Further, these methods cannot provide any information about the nature of the tumors. It is of particular interest because benign abnormalities are commonly found, particularly among those who had TB or other lung disease. Because of these two problems, photographic diagnosis often overdiagnose and requires highly stressful bronchoscopy and biopsy.

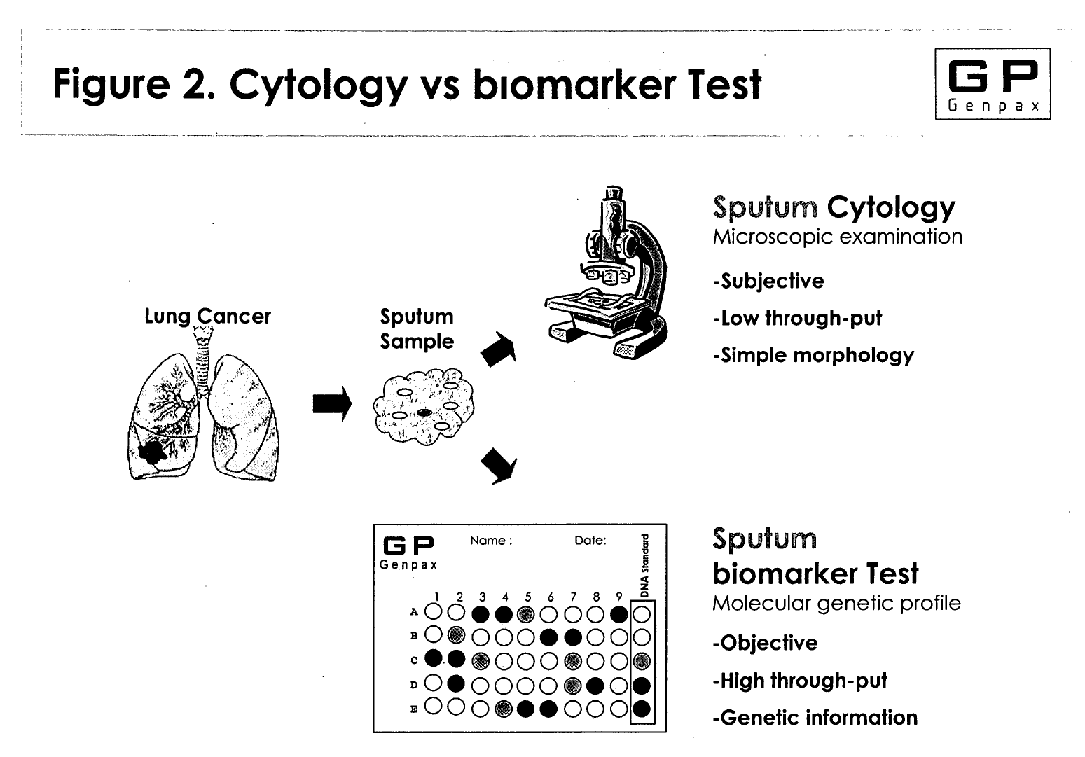

To address the limitations in conventional photographic methods, sputum cytology, as illustrated in FIG. 2, has been used in early diagnosis of lung cancer since 1950s. Despite the relatively small size of tumor, or small number of cancer cells in early stages, sputum samples often contain various lung samples, particularly the epithelium exfoliated by coughing. Squmous cell carcinoma, the most frequent type of lung cancer, occurs in the epithelium. However, as the method requires trained pathologist to examine each sample under microscope, it is prone to subjective judgment and have poor processivity. To improve the processivity maintaining efficiency in early diagnosis, the methodology is being renovated by automated biomarker test.

The present invention specifically recognizes these limitations of the conventional methodologies in the diagnosis of cancer and utilizes a novel in-situ screening method to identify over-expressed specific lung cancer genes as described in more detail below.

As a way of providing background, no single marker can diagnose all lung cancers, because lung cancer is a collective term for tumors of various pathogenic origins. In other words, there are many different kinds of lung cancers with independent causes and developmental pathways. Some grow fast but stay localized; some metastasize at early stages; some are responsive to certain drugs, while others resistant to them, and so on. Even in a given type of tumor, genetic background of the individual patients determines the variant magnitude of the clinical phenotypes. In sum, there are numerous types of lung cancers multiplied by various stages and familial backgrounds for each type. Theoretically, at least as many molecular markers or their combinations are needed to bring about the variant numbers of the clinical phenotypes. The present invention contemplates identifying these markers and using their combinatorial information of the gene expression profiles for lung cancer molecular genetic diagnostics.

In the past few decades biomedical scientists have invented ingenious methods to discover genes whose expression marks certain phenotypes. These methods can be largely divided into forward and reverse genetics; forward genetic methods are generally called the classic methods that search the mutated gene for an already identified trait. These methods often take enormous effort and time, therefore, rarely adopted for industrial purposes. Reverse genetic methods allow faster and easier gene discovery with some functional correlation depending on the design of the screening method. Most widely used reverse genetic method is differential screening, where the genes from two different samples are compared, and those genes that are differentially up- or down-regulated in the tissue of interest are selectively cloned. Though proven effective in many cases (29, 30), these methods have two major problems. One is the intrinsic bias toward the tissue of interest. Human lung cancer is a complex genetic disease originating from various cell types, developing into various dichotomy of carcinogenic pathways. If single specific type is used, makers for other types will be lost, and if many different types and/or stages are mixed, many lung! cancer markers such as p53 will be lost as it is up-regulated in around half of cases, and down-regulated in the other half. Furthermore, tumors with markedly elevated expression of most genes will be normalized in such a way to lower the threshold expression level to lose most of the markers, and for tumors with suppressed gene expression, to raise the threshold to produce numerous false positives. Therefore, the conventional methods do not allow comparative analysis of wide variety of types and stages. Secondly, a tumor is a mixture of cancer cells and normal cells, and because conventional differential screening methods take cumulative gene expression levels, cancer-cell specific expression is masked by the expression in non-cancerous cells within the sample, particularly the genes expressed in a small number of cells, therefore, masked by relatively massive background signals.

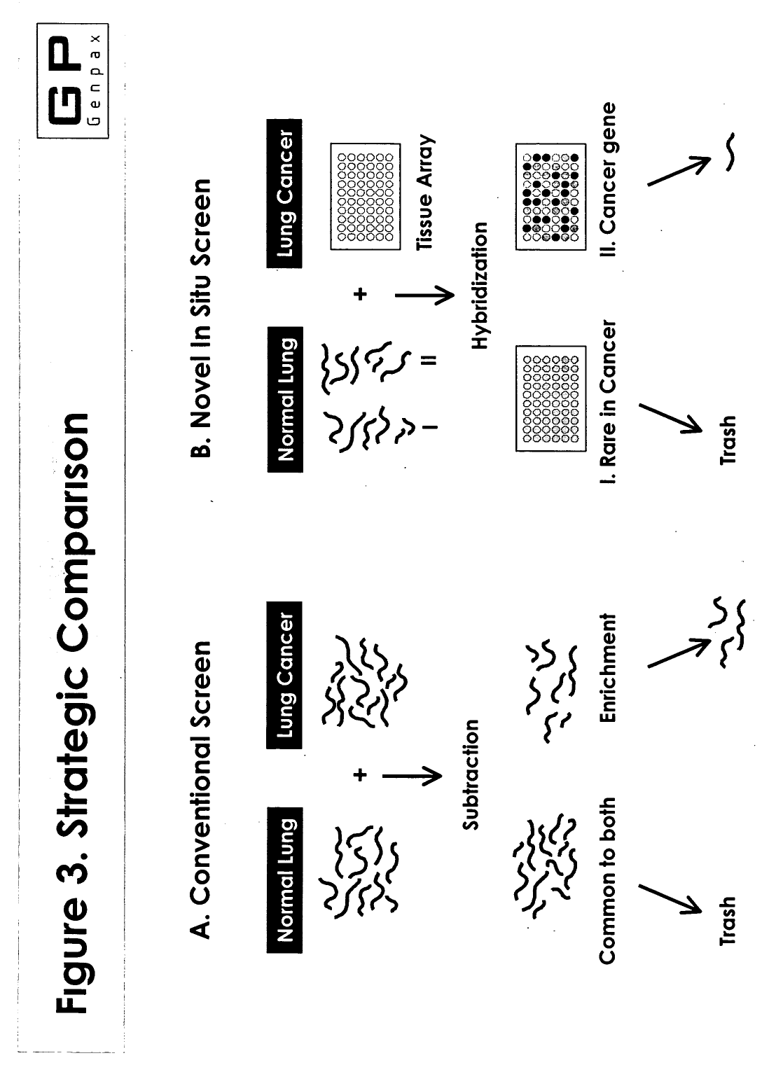

So, as illustrated in FIG. 3, in conventional differential screening, genes expressed in a tumor are subtracted by normal tissue that gave rise to the tumor to remove common genes. It is practically impossible to remove all the common genes, but this step simply enriches the gene pool for the cancer specific genes by selectively removing the common genes. If the subtraction is overdone, some of the cancer specific genes, particularly low expressors, are removed, and if underdone, majority will be false positives. In addition, depending on the types and/or the nature of the tumor the gene expression level varies in a great extent; some tumors exhibit elevated expression, whereas some show silencing the transcription machinery in general. In these cases, optimization of subtraction often fails. Even when the experiment turns out successful, two basic problems remain unresolved. First, it is impossible to determine what fraction of the target genes has been discovered. And more importantly, there are numerous types and kinds of tumors, and markers for one is often no longer a marker for the others.

Our novel in-situ screening strategy is not biased toward any specific type, nor masked by background signals. The present invention in one embodiment provides a method comprising in situ screening combined with tissue-arrays technology that allows examination of expression profiles of 240 genes in 60 different tumor samples in one experiment. More specifically, as also illustrated in FIG. 3, the present invention provides a method to screen 90% of all the cancer specific overexpressors for a various tumors of diverse nature. Estimated 30,000 human genes will be pooled by ten genes and therefore, three thousand pools are examined for the expression in tissue arrays with 60 different tumor samples of various kind and stages. The in situ gene expression pattern provides information regarding cancer cell specificity within a tumor, as well as cancer type specificity among various tumors without any bias. The present invention improves processivity of conventional in situ hybridization by at least few hundred folds as described in detail next.

Outline of the Procedures and Estimated Improvement

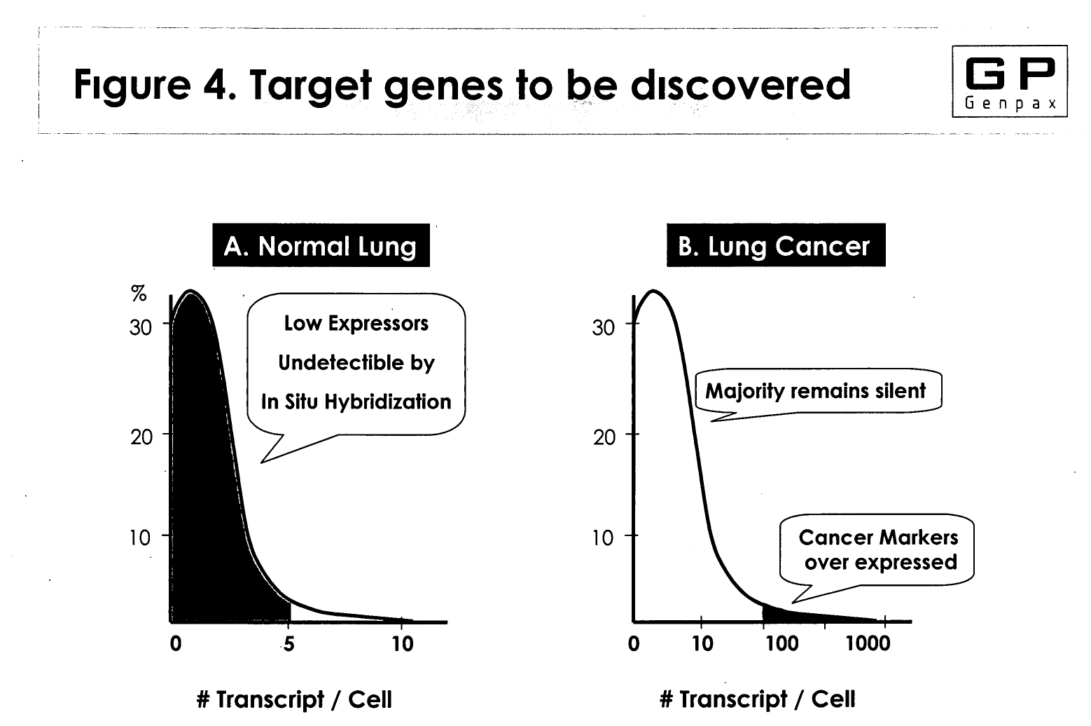

As illustrated in FIG. 4, one of the key elements in molecular diagnosis is cancer specific biomarkers. The present invention provides an efficient method of biomarker discovery. This diagram in FIG. 4 defines the target genes to be discovered and shows that: A. Around 90% of the genes expressed in normal lung are expressed at basal level with transcripts less than 5 per cell; and B. Among them are the target genes, as marked red, whose expression is activated as a consequence of cancer development. Cancer specific overexpression provides an excellent means to diagnose small number of cancer cells among a number of normal cells.

Non-radioactive in situ hybridization is not a very sensitive method to visualize gene expression patterns, unable to detect the basal level expression. This low sensitivity provide and excellent strategy to select the target genes: When combined, only the overexpressed genes produce gene expression patterns. Pools of ten clones contain average one overexpressed gene in the background of nine silent genes. The probability of losing a target gene due to mixing with a non-specific ubiquitous overexpressor is less than 9%, which has to be tolerated as the method improves processivity by 900%.

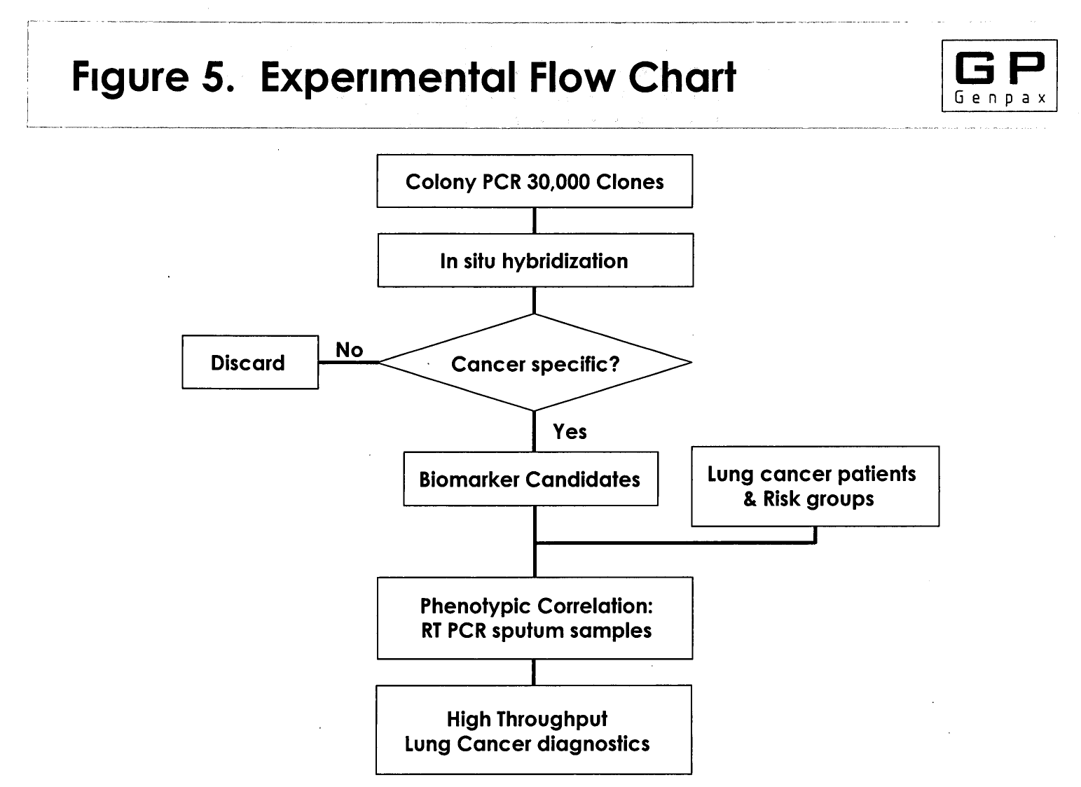

More specifically, the diagram in FIG. 5 provides a brief summary of experimental procedure in a flow chart format. To cover the human genome, 30,000 human genes (estimated gene number in human genome) is cloned/or purchased. Their gene expression patterns in various cancer samples are determined by in situ hybridization. Those showing cancer specific expression are collected as candidate lung cancer markers, and their efficacy in lung cancer diagnostic is confirmed by checking their expression in lung cancer patients by establishing correlation between phenotypes and the gene expression. Once strong correlation is established, their expression is to be used as a genomic marker for the phenotype. High throughput automated diagnostics can be developed utilizing these markers.

The method according to one embodiment of the present invention is established based on the following observations. First, in any given tissue including normal lung, about 10% or less of human genes are transcriptionally activated and more than 90% of the clones are transcribed in basal level (31, unpublished data). For the diagnostic purpose, basal transcription is not very useful due to low sensitivity due to the scarcity of the signal. Non-radioactive in situ hybridization is not sensitive enough to detect these low expressors, so when ten in situ probes are combined for one in situ hybridization experiment, average one expression pattern is detected. This turned out as an efficient strategy to selectively visualize the overexpressed 10% clones in the blank background of 90% low-expressors improving processivity almost 10 fold. To complete screening one thousand genes, hundred pools needs to processed, and around two clones (0.2%) are expected to give cancer specific gene expression patterns, therefore, two pools that contain these clones need to be individually processed. Therefore, a total of one hundred twenty in situ hybridization (12% effort) will cover one thousand clones.

Secondly, tissue array technology makes 60 different cancer samples arranged and sectioned together onto each histological slide, making one in situ hybridization worth 60 fold. Taken together with pools of ten in situ probes per in situ, and twenty four slides as unit to process, it becomes
(10 genes/slide)×(24 slides/unit)=240 genes per experimental unit
(30,000 human gene)/(240 genes/unit)+(60×10 individual genes)/(240 gene/unit)=128 experimental units to complete both pools and individual genes
[(128 units)/(1 week/unit/person)]/(52 weeks/year)=2.5 years by one person
[(30,000 genes)/(10 genes/slide)]/[(50 slides/tissue array block)×(2 tissue array blocks/60 tumors)]=180 tumors needed to complete.

This calculation is base on assumption of perfect experimental technique, and in reality it takes more time and effort. Unlike other technically sophisticated methods that requires in situ as final step to confirm cancer specific expression, this method generates the cancer specific expression patterns at the first step. In case a cancer-specific clone happens to be placed with one or more non-cancer-specific clones, its expression is masked by other expression pattern, and therefore, is lost. The probability of important clones to be lost in such a manner is around 8%. The estimated number of lung cancer specific genes are 30,000 genes×0.2%=60 genes, and around 55 of them are expected to be discovered by the present invention. Considering the economic merits of the method, this loss must tolerated.

### EXAMPLE 1

### Materials and Methods

Library Transformation

Library transformation was carried by first growing BM25.8 cell with shaking for overnight at 31° C. and then inoculating 200 ul of cultured cell to 2 ml of new LB broth and growing for 3 hour at 31° C. with vigorous shaking (200-300 rpm). Then, 20 ul of 1M MgCl2 were added and vortexed.

1 ul of library DNA to 9 ul SM buffer (1/10 dilution) was added to dilute library, and 1 ul of 1/10 diluted DNA was added with 200 ul of BM25.8 cell (of #3) followed by inoculating for 1 hour at 31° C. (with no shaking).

500 ul of LB both was added and an appropriate volume of transformed competent cells was transferred onto LB agar plate containing ampicillin. Finally, the plate was inverted and incubated at 31° C. for 12˜16 hour.

Plasmid Preparation

Plasmid was prepared by generally following Core-One manufacture's protocol which comprises the steps below:
(1) Pick a single colony and inoculate 2 ml of LB ampicillin (120 ug/ml). Grow approximately 12˜16 hrs, with shaking at 37° C.
(2) Harvest cells by centrifugation at 13000 rpm for 1 min at 4° C. Remove the supernatant.
(3) Resuspension: Add 250 ul of Cell resuspend solution. Resuspend cell fully.
(4) Disrupting cells: Add 250 ul of Cell Lysis solution. Mix by inverting the tube four times.
(5) Neutralization: Add 350 ul of DNA binding buffer and mix by inverting the tube 4 times.
(6) Centrifuge 13000 rpm for 15 min at 4° C.
(7) Transfer the cleared lysate to the spin column by decanting.
(8) Centrifuge the supernatant at 13000 rpm for 1 min at room temperature.
(9) Add 600 ul of Column Wash buffer to spin column.
(10) Centrifuge the supernatant at 13000 rpm for 1 min at room temperature. Remove the spin column from the tube and discard the flow through.
(11) Repeat the wash procedure using 300 ul of Column Wash buffer.
(12) Centrifuge the supernatant at 13000 rpm for 2 min at room temperature.
(13) Transfer the Spin Column to new 1.5 ml tube.
(14) Elute the plasmid DNA by adding 60 ul of distilled water.

Restriction Enzyme Digestion

Once the plasmid was prepared, enzyme digestion was performed by generally following the procedures below. First, to 20 ul of plasmid DND, the following were added: 0.3-1 ul of EcoRI (7˜20U); 3 ul of 10× buffer for EcoRI; 0.3 ul of 100×BSA; and up to 30 ul of distilled water. The solutions were mixed well and then incubated at 37° C. for 3 hours. Then, the pattern of cutting DNA in 0.9% agarose gel was checked.

Alternatively enzyme digestion was carried out by adding the following solutions: 20 ul of plasmid DNA, 0.3-1 ul of KpnI (7-20U), 3.5 ul of 10× buffer for KpnI, 0.35 ul of 100×BSA and up to 35 ul of distilled water. Again, the solutions were mixed well and then incubated at 37° C. for 3 hours. Then, the pattern of cutting DNA in 0.9% agarose gel was checked.

In Vitro Transcription

In vitro transcription was performed by generally following the procedures below. First, linear template DNA was prepared by digestion of superhelical plasmid DNA with a suitable restriction enzyme (EcoRI or KpnI). Then, the template DNA was purified by extraction with phenol:chloroform and standard precipitation with ethanol.

Then, the following components were mixed: 7.8 ul of DNA template; 1.5 ul of 10× buffer; 3 ul of Dig mix; 1.5 ul of 0.1 M DTT; and 0.2 ul of Rnase. To this mixture, 1 ul of T7 polymerase was added, and then incubated at 37° C. for 3 hours.

1.3% gel was then checked and 10U RNase-free DNase was added and the mixture was further incubated for 30 min at 37° C. Finally, the RNA was purified by extraction with phenol:chloroform and standard precipitation with ethanol.

In Situ Hybridization

In situ hybridization was then performed by generally following the procedures below. First, slides were dried for 1 day at 45° C.

Waxing and Rehydration:

Waxing and Rehydration was performed as follows:

| | |
|---|---|
| Xylen 1 | 10 minutes |
| Xylen 2 | 10 minutes |
| 100% Ethanol | 2 minutes |
| 95% Ethanol | 2 minutes |
| 80% Ethanol | 2 minutes |
| 70% Ethanol | 2 minutes |
| 40% Ethanol | 2 minutes |
| 2x SSPE | 2 minutes |

Refixation & Prehybridization:

Following the waxing and rehydration, refixation was carried out with 4% Paraformaldehyde(PFA) in PBS at room temperature for 15 minutes. Then, prehybridization was carried out by: rinsing the slides in 2×SSPE for 5 minutes; incubating the slides in 3 ug/ml Proteinase K at 37° C. for 30 minutes; again rinsing the slides in 2×SSPE for 5 minutes; incubating the slides in MEMFA at RT for 10 minutes; rinsing the slides in 2×SSPE for 5 minutes; incubating the slides in 0.2M HCl at room temperature for 15 minutes; rinsing the slides in 2×SSPE for 5 minutes; adding AP 1 buffer with Levamisole at 37° C. for 20 minutes; rinsing the slides in 2×SSPE for 5 minutes; adding 600 ul of hybridization buffer to each slide and then incubating in a humid chamber (50% formamide: 50% DW) at 65° C. for 2˜6 hours.

Hybridization:

Hybridization is then performed. Excess hybridization buffer was drained off and a piece of broken coverslip was placed at either end of the slide. 95 ul of 0.5 ug/ml probe solution was added to each slide, and a large coverslip on top of the sections and broken cover slips was placed to prevent evaporation of the probe solution. Then, incubation in a humid chamber at 60° C. overnight was provided.

Post Hybridization:

Post hybridization, the slides were soaked in 2×SSPE until the cover slips fall off, and 300 ul hybridization buffer was added and incubated at RT for 5 minutes. The slides were drained and 300 ul 50% hybridization buffer: 50% 2×SSPE: 0.3% CHAPS, was added followed by incubation at room temperature for 10 minutes.

Again the slides were drained and 500 ul 2×SSPE: 0.3% CHAPS were added. The slides were then soaked in 2×SSPE for 20 minutes and then in 50% Formamide: 50% 2×SSPE for 30 minutes at 50° C. The slide were then rinsed 5 times in PBSw for 10 minutes each, and 500 ul Antibody buffer were added to each slide for 2 hour at room temperature. Then mixed pre-block the antibody (anti-Dig AP 1:1000) in antibody buffer at 4° C. with gently rocking.

The slides were drained and 200 ul of pre-block the antibody were added for overnight at 4° C. The slides were rinsed 3 times in 0.1% BSA in PBSw for 10 minutes each wash and then in AP1 buffer for 10 minutes.

Staining was carried out by adding enough BM purple for 4° C. and washing 2 times in PBSw for 10 minutes and soaking in MEMFA for 30 min.

Dehydration was then performed as follows:

| | |
|---|---|
| 2x SSPE | 2 minutes |
| 40% Ethanol | 2 minutes |
| 70% Ethanol | 2 minutes |
| 80% Ethanol | 2 minutes |
| 95% Ethanol | 2 minutes |
| 100% Ethanol | 2 minutes |
| 100% Methanol | 2 minutes |
| Xylen 2 | 10 minutes |

Then the slides were mounted with Permount:Xylen=1:1 and dried.

Solutions

The following solutions were utilized.

Hybridization solution (for 1 L): 10 g of Boehringer Block; 500 ml Formamide; 250 ml 20×SSC; heat at 65° C. for 2 hours; 120 ml DEPC water; 100 ml Torula RNA (10 mg/ml in water; filtered); 2 ml Heparin (50 mg/ml in 1×SSC); 5 ml 20% Tween-20; 10 ml 10% CHAPS; and 10 ml 0.5 M EDTA.
20×SSPE (for 1 L): 175.3 gNaCl; 27.6 gNaH2PO4; 7.4 g EDTA; and 800 ml DDW.
PBSw: PBS with 0.1% Tween-20
Antibody buffer: 10% Heat inactivated Goat Serum; 1% Boehringer Block; 0.1% Tween-20; and dissolve in PBS at 70° C.
AP1 buffer: 0.1 M NaCl; 0.1 M Tris pH 9.5; 50 mM MgCl2; and add Levamisol 0.025 g/100 ml.
IX MEMFA: 100 mM MOPS (pH 7.4); 2 mM EGTA; 1 mM MgSO4; and 20% Formaldehyde.
Lung cancer tissue array: Tissue array blocks are arranged reflecting the frequencies of types and stages of lung cancer in Korea as removed from surgery; 60% are squmous cell carcinoma, the most frequent type found among smokers, and 25% adenocarcinoma, 12% small cell lung cancer, 3% large cell carcinoma. Tissue arrays are manufactured by Superbiochip Inc. with the cancer samples provided by Seoul National University School of Medicine, Dept. of Thoracic Surgery.
In situ probes: Probes are Synthesized as Described Above.

Results

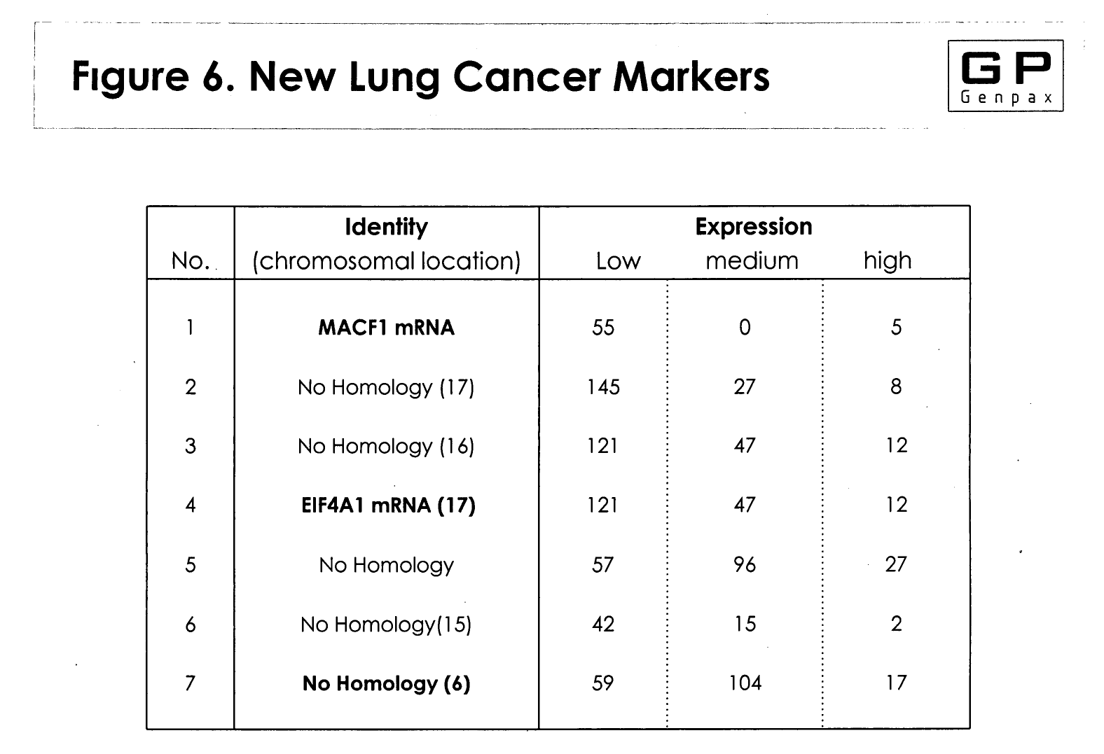

DNA from a normal lung library (Clontech) was transformed into bacterial cell line BM25.8 that circularize the clones into plasmid forms, plated onto culture medium and resulting single clones were randomly picked and grown up. From each clone, plasmids were prepared and subjected to restriction enzyme analysis. 8,270 individual clones were picked and analyzed by restriction enzyme analysis. Colony PCR was carried out to amplify inserts and T7 promoter sequence required to drive antisense mRNA production. 2940 clones are prepared in this manner and the results were compared with those from the plasmid preps. For a first set of in situ hybridization, we have chosen to work with 3,160 clones that show insert size over 5 kilobases. 10 clones were pooled to be transcribed and the generated probes were hybridized on tissue array slide containing 60 different lung cancer tissue samples. Therefore, 316 in situ hybridization experiments were performed. Seven of those pools showed positive signal in at least one cancer type tissue but non-detectible level in normal tissues, and four additional pools with ubiquitous expression were selected to investigate the possibility of cancer specific gene expression being masked by the evident expression. To identify individual clones, 120 (110 individual clones+one pool (10 clones) repeat) additional in situ experiments were performed to trace back which clones are responsible for the positive signals. Four pools with the ubiquitous expression did not contain hidden expression, in all tissue sections, indicating that those pools contain either housekeeping genes or genes that are not specific to certain cancer type. Seven clones showed certain cancer type specific pattern in the in situ experiments. The table in FIG. 6 shows the list of the discovered lung cancer markers and their identity as determined by sequence analysis and Blast search in NCBI database (32).

The following seven positive clones, SEQ Nos. 1-7 were identified as shown below and also provided in the sequence listing.

Clone #1 Sequence (5′-->3′)

ACTACATTGAGCATGATGTGTCTCCTGAATGTGTGTTTCATGTGAAGTA
TTGTTCTGATTAACTGACATCCTTGCTTACAAGTTTCACTAACCCTTTGGAGTT
TAAGCACAAATGCACAAAGGGAAAAGAGGACGACCTGTTTGGGGTTCTTTTT
TGCAAAAACAAACAGTCGCATGCTGGACGCTAACACCAAGCTTACACTGTGT
GTGTGATACGGCTGAGCTGCTCCATAAGGCTCTATCTTTTATCTGCCCAAGGC
GTGCCCTGCAACTCTGGAATGCAGAGCAGTTGCTGGGGTGATTGACCTAGGC
ACAGTGGAGATATTTCCCATCTTCAAAGCCATGCAAAAGGGCCTCCTTGACC
AAGACACAGGCCTAGTGCTTCTGGAATCTCAGGTTATCATGTCTGGCCTCATT
GCCCCTGAGACGGGTGAAAACCTCTCTTTGGAGGAGGGCGTAGCCAGAAACC
TCATTAATCCCCAGATGTACCAGCAGCTCCGGGAGCTACAGGAGCCCTGGCC
TTAATAAGCAGGCTTACTGAGAGCAGAGGCCCTCTTTCTGTGGTGGAAGCAA
TTGAAAAGACAATAATCAGTGAGACAGTTGGACTGAAAATCTTAGAAGT

Location: Homo sapiens chromosome 16
Homology: Homo sapiens macrophin 1 isoform 4 (MACF 1) mRNA
Identities=344/347 (99%)

Clone #2 Sequence (5′-->3′)

GTGGTGGTGGGCGCCTGTAATCCCAGCTACTTGGGAGGCTGAGGCAGA
GAACTGCTTGAACCCAGGAGGCAGAGGTTACAGTGAGCCAAGATCGCACCA
CTGCACTCCAGCCTCCAGCCTTCAGCCTTGGTGACAGAGCAAGACTCTGTCTC
AAAAAGAAAGAAAAAGAAAAAGACTGTGAAAGAACACACATCAAAATGTTA
AGCAGTGGTTTGTATCTTGAAGGACAATTTTTTTTTATTGGAATGTTTCTTCTC
TATATTTTTGG

Location: Homo sapiens chromosome 17
Identities=152/152 (100%)
Homology: None reported

Clone #3 Sequence (5′-->3′)

CGGGCCCGGGATGGACTGAACCAAGACCAGCAGCCAACTTAGAGGCT
CAGTTTTAAGGCCTTGACTTGGGATAGTAAGATTAGAGATTTCCAGCAGTGTC
TCCTCCCCGCACCTCCCCCCACCCCCCCGCCCCCCGCTTTTTAGTGAAGAGAA
AGTCACATAAAGATAACCATTTAAAAGTGAGTAATTCAAGGCCAGGCGCGGT
GGCCCATGCCTGTAATCCCAGCACTTTGGGCGGCTGAGGCAGGTGGATCACT
TGAGGTTAGGAGTTCGAGCCCAGCCTGGTCAACATGGTGAAACCCCGTCTCT
ACTAAAAATATAAAAATTAGCCGGGTGTGGTGGCAGGCACCTGTAATCCCAG
CTATTAGGGAGGCTGAGGCAGGAGAATTGCTTGAGCCTGGGAGGCAGAGGTT
GCAGCGAGCCAAGATTGTGCCACTGTACTCCAGCCTGAGCGACGGAGCGAGA
ATCTGTCTCAAAAAAAAAAAAAGATAATTCA

Location: Homo sapiens chromosome 16
Identities=339/339 (100%)
Homology: None reported

Clone #4 Sequence (3′-->5′)

AGTTTCTAAGGATCATGTCTGCGAGCCAGGATTCCCGATCCAGAGACAATGG
CCCCGATGGGATGGAGCCCGAAGGCGTCATCGAGGTGAGACTGGAGAAATG
GAATTCTGTCCTCCCCCATTACAACTTTCAGCCGTATAGAGTTAGAGTGGCCT
CTTGATTGATTTCCCAGATCATCTAGAAGCAGCTGGTTTCCCTAAAGGGAGGA
GGGTTGTAAGCTCTGAGGCTTTTGTTAGTAGGCACCAGATTCTGTTTGCTCGG
AGACTACAGCTCAGCTCCACCTTTTCCATGACTCAAGCTTTAATTTCTTTGCAT
CCCCTAGAGTAACTGGAATGAGATTGTTGACAGCTTTGATGACATGAACCTCT
CGGAGTCCCTTCTCCGTGGCATCTACGCCTATGGTTTTGAGAAGCCCTCTGCC
ATCCAGCAGCGAGCCATTCTACCTTGTATCAAGGGTGAGACCTCTCAGTCCC
AGAAGACATTGTGGACTGTCCCTGACCTGGGTAGAGTGGCATCTGGTTGGTG
ATGCCCATCTCATATCAGCCAGGGACAAAGCAACTCCTTGTTCATCCCAGCTT
GGCTTTTGATCCGTGCCCATGCCTGGTTCATGCCTTGGACACATAGGTTTCCT
TTAAAGAGGTGGTATTGTAGCCAGCTTATATTTGCATCTATAGCCATGTTTCT
AGTCCAGCTTGGTGTGCAATACTAGATGAGTTAATAACTGGTCCTTGTTTCTG
ATCTGGTTCCCATTGTGTAACTGTGTTGATTGGG

Location: Homo sapiens chromosome 17
Identities=734/736 (99%)
Homology: eukaryotic translation initiation factor 4A, isoform 1 (EIF4A1)
Identities=137/137 (100%)

Clone #5 Sequence (5′-->3′)

GCCTTATGGCCGGGGACAACCTTAGCCAACCATTTACCCAAATAAAGT
ATAGGCGATAGAAATTGAAACCTGGCGCAATAGATATAGTACCGCAAGGGA
AAGATGAAAAATTATAGCCAAGCATAATATAGCAAGGACTAACCCCTATACC
TTCTGCATAATGAATTAACTAGAAATAACTTTGCAAGGAGAGCCAAAGCTAA
GACCCCCGAAACCAGACGAGCTACCTAAGAACAGCTAAAAGAGCACACCCG
TCTATGTAGCAAAATAGTGGGAAGATTTATAGGTAGAGGCGACAAACCTACC
GAGCCTGGTGATAGCTGGTTGTCCAAGATAGAATCTTAGTTCAACTTTAAATT
TGCCCACAGAACCCTCTAAATCCCCTTGTAAATTTAACTGTTAGTCCAAAGAG
GAACAGCTCTTTGGACACTAGGAAAAAACCTTGTAGAGAGAGTAAAAAATTT
AACACCCATAGTAGGCCTAAAAGCAGCCACCAATTAAGAAAGCGTTCAAGCT
CAACACCCACTACCTAAAAAATCCCAAACATATAACTGAACTCCTCACACCC
AATTGGACCAATCTATCACCCTATAGAAGAACTAATGTTAGTATAAGTAACA
TGAAAACATTCTCCTCCGCATAAGCCTGCGTCAGATTAAAACACTGAACTGA
CAATTAACAGCCCAATATCTACATCAACCAACA

Location: Not determined
Homology: None reported

Clone #6 Sequence (5′-->3′)

TGGCTCATGGCTACAATCCCAGCACTTTGGGAGGCCGAGGCAGGCAGATCAC
CGGAGGTCAGGAGTTCAAGACCAGCCTGACCAACACGGAGAAACCCCGTCC
CAACTAAAAATACAAAATTAGCCAGGCATGGTGGCACATGCCTGTAATACCA
GCTACTCAGGAGGCTGAGGCAGGAGAATGACTTGAACCTGAGAGGCAAATG
CTGCAGTGAGCCGAGATCAGGCCATTGCACTCCAGCCTGGGAAACAAGAGGC
AAAACTCCGTCTCCAAAAAAAAAAAAAAAAAAAAAAAAAAAAA

Location: Homo sapiens chromosome 15
Identities=264/272 (97%)
Homology: None reported

Clone #7 Sequence (5′-->3′)

GATATGAAATGACTCCCTCAGACAATTTTTAAAAAGATAAGTTTTTTA
AAGACCAATAAAAACCTAAGGGACAAAATAAGACATTGATGATTTGAAATTT
CTTTGTAACAAAATATACTATAAATTTGAAAGCAAAGGATAGACTGGAAGAG
AATATTTGCAATATTTAAAACAGGCTAATGGTCAGTGCTCACAATATATAAC
ATGCTCTCATGTATCAATTTTAAAACAACACCCTTGTAAAAAAAAAAAAAAA
AAGGATCCAATGAGGCAGGGTACAAATAACAAATTCTTAGCAAAATAATTTA
GCTCCTGAAATGATACTCATTCTTACTGGAAACCAGGGGAATGCANATTCTA
ATAGGTTATTTTTTTTGCTTATGAAATTTGCAANAATAAAAGNGACTACTGAG
CTTCNTTTTTTGTAANAGNGTAGNGAAACTAGTATCTGCATNCCCNGTNGGG
GATGGTATAAATTGGCACAGTATTTTTTACATTAGNGCATTGATGTATTTTTA
AAACACTTATATATTGCACAATTATCAAATCTGCACAGCAGTTTTTATTTGAT
AATCTGTTCTACAAAAATACTCATAAAGGACACAAATATAAGGAATTACATC
ATTAATTATTATCAATTCCCATGNAGCCATTTAAAAGCATTTNGGGGGATCTC
TATGGAAATTGGCATGGAATTATTTNATTTCANAAAAATTATTTTTTTAATCC
ATGGAANCCTTGGATACTGGNTTGCGGGGCTTGAAAAACTTCTTCCAAGAAA
ATTNTTTATTTGGGAAAAAAAATTAAAGGNAAAAATTTGGGAATTAAATAAG
GGANTTCCATNATAAGGGANGGGTAAAAACCTAAAAAAGCCNGGGTNGGGG
GNATTTTTAATNGGGGGTTAANNGGGGGATTACNATTTGGNAAAAANTTTGG
NAANGGGGNTTTTTNTT

Location: Homo sapiens chromosome 6
Identities=408/449 (90%), Gaps=5/449
Homology: None reported.

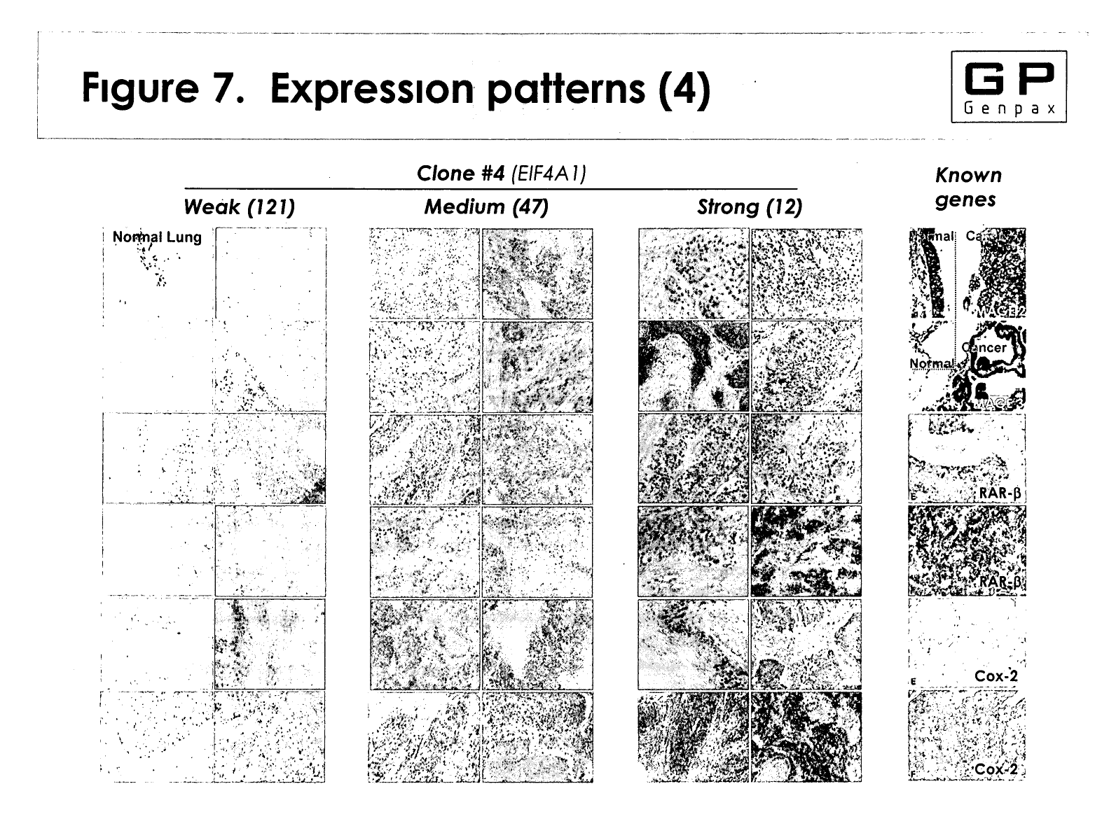

Seven positive clones, SEQ Nos. 1-7 identified were subjected to extensive in situ analyses using additional clinical lung tissue samples to confirm their lung cancer tissue specific expression. At least 180 different cancer samples are being tested for each of the seven positive clones. The nucleotide sequences have been determined and analyzed. Six of the seven genes shows sequence match in Human Genome Database, and two with known functional human mRNA sequences as human macrophin 1 isoform 4 (MACF1) mRNA and Eukaryotic translation Initiation Factor 4A isoform 1 (EIF4A1), mRNA. Particularly, cancer specific overexpression of translation initiation factor EIF4A1 might be necessary for the elevated production of cellular building blocks in a highly proliferative state. Investigation of the mechanism underlying activation of EIF4A1 might generate important information on the cellular mechanism of cancer specific proliferation, and possibly an effective means to interfere with it. Interestingly, as shown in FIG. 7, EIF4A1 expression level in clone having SEQ. No. 4 varies in a wide spectrum of tumor samples as the expression levels are arbitrarily classified as weak, medium, to strong. As indicated in the parenthesis 121 of 180 tumors tested show weak expression, 47 show medium level, and 12 strong expression indicating that there exist variety of cancerous metabolism.

Among the seven clones, clones having SEQ. Nos. 3 and 4 showed very similar expression patterns and were coincidently found in pool #38. The rest were all found independent from each other as well as from ubiquitously expressed genes. Clone having SEQ. No. 1 is highly specific in a very small number of cells that resemble developing vasculature, and clone 17 in squmous cell carcinoma, 3 and 4 in squmous cell carcinoma too, 5 in squmous cell carcinoma and adenocarcinoma, 6 in squmous cell carcinoma, and 7 in almost all different types of lung cancer. All these markers showed weak or undetectable level of expression in normal lung tissue. The actual sequences of the seven identified are provided in a sequence listing attached hereto.

Correlation analysis is in progress to establish an algorithm that connects EIF4A1 expression level as well as other genes to clinical phenotypes such as growth rate, invasion, or even death/survival rate.

A Composition Comprising the Over-Expressed Genes Specific to Lung Cancer Cells:

In additional embodiments, the present invention concerns compositions comprising one or more of the polynucleotides disclosed herein to be suitable for the diagnostic applications of the present invention.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5.times.SSC, 0.5% SDS, 1.0 mM EDTA (pH 8.0); hybridizing at 50.degree. C.-65.degree. C., 5.times.SSC, overnight; followed by washing twice at 65.degree. C. for 20 minutes with each of 2.times., 0.5.times. and 0.2.times.SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Any polynucleotide that encodes a lung tumor protein or a portion or other variant thereof as described herein is encompassed by the present invention. Preferred polynucleotides comprise at least 15 consecutive nucleotides, preferably at least 30 consecutive nucleotides and more preferably at least 45 consecutive nucleotides that encode a portion of a lung tumor protein. More preferably, a polynucleotide encodes an immunogenic portion of a lung tumor protein. Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

It will also be understood that, if desired, the nucleic acid segment, RNA or DNA compositions that express a polypeptide as disclosed herein may be used in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be used along with various other agents as required in the particular instance. Such compositions may be purified from host cells or other biological sources, or alternatively may be chemically synthesized as described herein. Likewise, such compositions may further comprise substituted or derivatized RNA or DNA compositions.

Molecular Classification and Diagnostic Applications:

The present invention further concerns compiling the profiles of the lung-cancer-overexpressed genes which can be used as the primary tools for molecular classification and diagnostics for different lung cancer types.

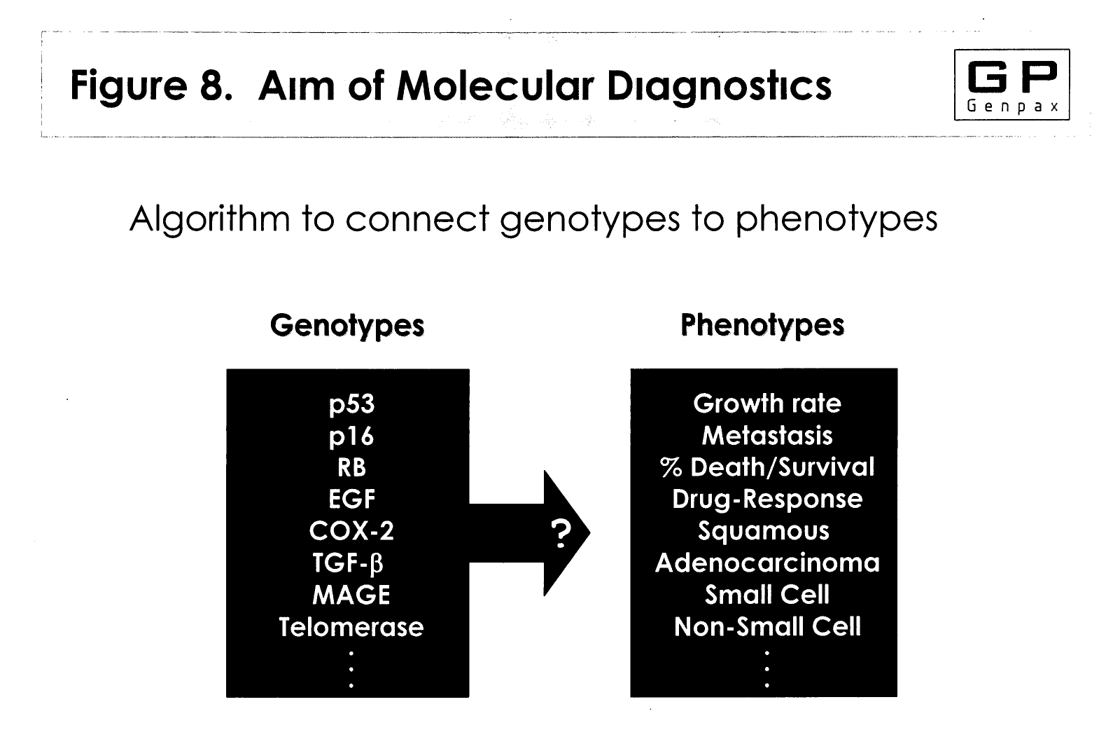

The present invention still further concerns an algorithm that connects the gene expression profiles to the clinical phenotypes, and a high throughput diagnostic methodology based on detecting a lung tumor protein, or mRNA encoding such a protein, in a sample. As shown in FIG. 8, molecular diagnostics is based on the algorithm that connects both morphological and clinical phenotypes to genotypes such that overexpression of a specific gene or gene set provides parameters for growth rate, metastatic nature, responsiveness to various drugs, cancer types.

The present invention further provides, within other aspects, methods for determining the presence or absence of a cancer in a patient, comprising the steps of: (a) contacting a biological sample obtained from a patient with an oligonucleotide that hybridizes to a polynucleotide that is identified as lung tumor specific; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient.

In related aspects, methods are provided for monitoring the progression of a cancer in a patient, comprising the steps of: (a) contacting a biological sample obtained from a patient with an oligonucleotide that hybridizes to a polynucleotide identified as lung cancer specific; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Those skilled in the art will readily appreciate that the present invention is adapted to carry out the objects and obtain the ends and advantages mentioned, as well as those inherent therein. The methods, compositions and use described herein are presently representative, preferred embodiments, are exemplary, and are not intended as limitations on the scope of the invention. Changes and modifications will occur to those skilled in the art upon reading this specification. It is understood that any and all of such changes and modifications are encompassed within the scope of the invention.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with any proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### REFERENCES

1. Ries L., Eisner M., Kosary C., Hankey B., Miller B., Clegg L., et al. SEER cancer statistics review, 1973-1997. Betheda, Md.: National Cancer Institute, 2000
2. Lorenso D., Andrea I., Francesca R., Alberto O., Grazia T., and Massimo P. Stage I Nonsmall cell lung carcinoma; Analusis of survival and implications for screening. CANCER Supplement 2000; 89(11) 2334-2344
3. Naruke T, Tsuchiya R, Kondo H, Asamura H, Nakayama H. Implications of staging in lung cancer. Chest 1997; 112:242s-8s
4. Adebonojo S A, Bowser A N, Moritz D M, Corcoran P C. Impact of revised stage classification of lung cancer on survival: a military experience. Chest 1999; 115:1507-13
5. Harpole D H, Herndon J E, Young W G, Wolfe W G, Sabiston D G. Stage I non-small cell lung cancer: a multivariate analysis of treatment method and patterns of recurrence. Cancer 1995; 76: 787-97
6. Strauss G M, Kwiatkowski D J, Harpole D H, Godleski J J, Richards W G, Herndon J E, et al. Extent of surgery influences prognosis in stage I non-small cell lung cancer (NSCLC): implications for treatment and screening for lung cancer. Chest 1997; 12: 97S
7. Wada H, Tanaka F, Yanagihara K, Ariyasu T, Fukuse T, Yokomise H, et al. Time trends and survival after operations for primary lung cancer from 1976 through 1990. J Thorac Cardiovasc Surg 1996; 112: 349-55
8. Williams D E, Pairolero P C, Davis C S, Bernatz P E, Payne W S, Taylor W F, et al. Survival of patient surgically treated for stage I lung cancer. J Thorac Cardiovasc Surg 1981; 82: 70-6
9. Warren W H, Faber L P. Segmentectomy versus lobectomy in patients with stage I pulmonary carcinoma. Five-year survival and patterns of intrathoracic recurrence. J Thorac Cardiovasc Surg 1994; 107: 1087-94
10. Martini N, Rusch V W, Bains M S, Kris M G, Flehinger B J, Ginsberg R J. Factors influencing ten-year survival in resected stages I to IIIA non-small cell lung cancer (discussion). J Thorac Cardiovasc Surg 1999; 117: 32-8
11. Papanicolaou G N, Koprowska I. Carcinoma in situ of right lower bronchus: Case Report. Cancer 1951; 4: 141-6
12. Umiker W, Storey C. Bronchogenic carcinoma in situ: report of a case with positive biopsy, cytological examination and lobectomy. Cancer 1952; 5: 369-71
13. Lemer M A, Rosbash H, Frank H A, Fleischner F G. Radiologic localization and management of cytologically discovered bronchial carcinoma. N Engl J med 1961; 264: 480-5
14. Lolman C V, Okinaka A. Occult carcinoma of the lung. J Thorac Cardiovasc Surg 1964; 47: 466-71
15. Pearson F G, Thompson D W. Occult carcinoma of the bronchus. J Can Med Assoc 1966; 94: 825-33
16. Woolner L B, Anderson H A, Bernatz P E. Occult carcinoma of the chonchus: A study of 15 cases of in situ or early invasive bronchogenic carcimona. Dis Chest 1966; 37: 278-88
17. Fullmer C D, Parrish C M. Pulmonary cytology: a diagnostic method for occult carcinoma Acta Cytol 1969; 13: 645-51
18. Meyer J A, Bechtold E, Jones D B. Positive sputum cytologic tests for five years before specific detection of bronchial carcinoma. J Thorac Cardiovasc Surg 1969; 57: 318-24
19. Bell J W. Positive sputum cytology and negative chest roentgenograms: A surgeon's dilemma. Ann Thorac Surg 1970; 9: 149-57
20. Marsh B R, Frost J K, Erozan Y S, Carter D. Occult bronchogenic carcinoma. Cancer 1972; 30: 1348-52
21. Melamed M R, Koss L G, Clifton E E. Roentgenlogically occult lung cancer diagnosed by cytology. Cancer 1963; 16: 1537-51
22. Martini N, Melamed M R, Clifton E E. Occult lung cancer diagnosed by cytology. Clin Bull Memorial Sloan-kettering Cancer Center 1971; 1: 107-10
23. Martimi N, Beattie E J Jr, Clifton E E, Melamed M R. Radilolgically occult lung cancer. Report of 26 cases. Surg Clin N Amer 1974; 54: 811-23
24. Valle R P, Chavany C, Zhukov T A, Jendoubi M. New approaches for biomarker discovery in lung cancer. Expert Rev Mol Diagn. 2003 Jan.; 3(1):55-67.
25. Mulshine J L, De Luca L M, Derick R L, Tockman M S, Webster R, Placke M E. Considerations in developing successful, population-based molecular screening and prevention of lung cancer. Cancer 2000 Dec.; 1(89):2465-7
26. Brambilla C, Fievet F, Jeanmart M, De Fraipont F, Lantuejoul S, Frappat V, Ferretti G, Brichon P Y, Moro-Sibilot D. Early detection of lung cancer: role of biomarkers. Eur Respir J Suppl. 2003 Jan.; 39:36s-44s
27. Field J K, Brambilla C, Caporaso N, Flahault A, Henschke C, Herman J, Hirsch F, Lachmann P, Lam S, Jaier S, Montuenga L M, Musshine J, Murphy M, Pullen J, Spitz M, Tockman M, Tyndale R, Wistuba I, Yongson J. Consensus statements from the Second International Lung Cancer Molecular Biomarkers Workshop: a European strategy for developing lung cancer molecular diagnostics in high risk populations. Int J Oncol. 2002 21(2):369-73
28. Srivastava S, Kramer B S. Genetics of lung cancer: implications for early detection and prevention. Cancer Treat Res. 1995;72:91-110.

1. A method of identifying cancer specific marker genes, comprising:
preparing clones by transforming DNA of interest into plasmids, said plasmids each containing a single clone;
providing a tissue array containing multiple cancer tissue samples;
providing a pool of said clones and hybridizing said clones with said cancer tissue samples on said tissue array; and
screening and identifying clones displaying positive hybridization with said cancer tissue samples.

2. The method of claim 1, wherein said cancer is lung cancer.

3. The method of claim 1, wherein said multiple cancer tissue samples each contains a different type of cancer tissue.

4. The method of claim 2, wherein said clones displaying positive hybridization with said cancer tissue samples have any of the polynucleotide sequences provided in SEQ Nos. 1-7.

5. The method of claim 1, further comprising identifying one or more clinical phenotypes expressed by each of said clones.

6. The method of claim 5, wherein said phenotypes include cancer histotypes, developmental stages, responsiveness to various therapies, and survival/death rates.

7. A method of identifying genes overexpressed in lung cancer tissues, comprising:
preparing candidate lung cancer specific clones by transforming DNA of interest;
providing tissue samples of lung cancer;
providing a pool of said clones and hybridizing said clones with said tissue samples; and
determining whether said pool of said clones positively hybridizes with said cancer tissue samples; and
screening and identifying each individual clone responsible for positive hybridization.

8. The method of claim 7, wherein said clone has any of the polynucleotide sequences provided in SEQ Nos. 1-7.

9. The method of claim 7, further comprising identifying clinical phenotypes expressed by said clone.

10. The method of claim 9, wherein said phenotypes include cancer histotypes, developmental stages, responsiveness to various therapies, and survival/death rates.

11. A method of detecting a cancer in a patient, comprising:
contacting a biological sample obtained from a patient with an oligonucleotide that hybridizes to a polynucleotide that is identified as lung tumor specific;
detecting in the sample a level of a polynucleotide that hybridizes to the oligonucleotide; and
comparing the level of polynucleotide that hybridizes to the oligonucleotide with a predetermined cut-off value, and therefrom determining the presence or absence of a cancer in the patient.

12. The method of claim 11, wherein said cancer is lung cancer.

13. The method of claim 11, wherein said polynucleotide that is identified as lung tumor specific is identified according to the method of claim 1 or claim 7.

14. A method of monitoring the progression of a cancer in a patient, comprising:
(a) contacting a biological sample obtained from a patient with an oligonucleotide that hybridizes to a polynucleotide identified as lung cancer specific;
(b) detecting in the sample an amount of a polynucleotide that hybridizes to the oligonucleotide;
(c) repeating steps (a) and (b) using a biological sample obtained from the patient at a subsequent point in time; and
(d) comparing the amount of polynucleotide detected in step (c) with the amount detected in step (b) and therefrom monitoring the progression of the cancer in the patient.

15. The method of claim 14, wherein said cancer is lung cancer.

16. The method of claim 14, wherein said polynucleotide that is identified as lung tumor specific is identified according to the method of claim 1 or claim 7.
